(19) United States
(12) Patent Application Publication
(10) Pub. No.: US 2012/0203286 A1
(43) Pub. Date: Aug. 9, 2012

(54) Bone Fastener and Methods of Use

(75) Inventors: Rex W. Armstrong, Cordova, TN (US); Bryan S. Wilcox, Collierville, TN (US); Julien J. Prevost, Memphis, TN (US); James Michael Mirda, Cordova, TN (US); Joshua W. Simpson, Collierville, TN (US); Robert M. Loke, Memphis, TN (US)

(73) Assignee: WARSAW ORTHOPEDIC, INC., Warsaw, IN (US)

(21) Appl. No.: 13/021,390

(22) Filed: Feb. 4, 2011

(51) Int. Cl. A61B 17/86

(52) U.S. Cl. 606/304; 606/305

(57) ABSTRACT

A bone fastener includes a shaft extending from a first end to a second end and defines a longitudinal axis. The shaft includes an inner surface that defines an axially extending cavity. The bone fastener further includes a body disposed within the cavity and in fixed engagement with the inner surface. The body includes a rigid element positioned within only a subregion of the cavity to form a stiffened zone exclusively in an adjacent portion of the shaft. Methods of use are disclosed.

### TECHNICAL FIELD

The present disclosure generally relates to medical devices for the treatment of bone disorders, and more particularly to a bone fastener configured to reduce stress in a vertebral rod system and increase fastener durability.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders include discectomy, laminectomy, fusion and implantable prosthetics. As part of these surgical treatments, spinal constructs such as vertebral rods are often used to provide stability to a treated region. Rods redirect stresses away from a damaged or defective region while healing takes place to restore proper alignment and generally support the vertebral members. During surgical treatment, one or more rods may be attached via fasteners to the exterior of two or more vertebral members. This disclosure describes an improvement over these prior art technologies.

### SUMMARY OF THE INVENTION

Accordingly, a bone fastener is disclosed that is configured to reduce stress in a vertebral rod system and increase fastener durability with an improved fastener/bone interface.

In one particular embodiment, in accordance with the principles of the present disclosure, a bone fastener is provided. The bone fastener includes a shaft extending from a first end to a second end and defining a longitudinal axis. The shaft includes an inner surface that defines an axially extending cavity. The bone fastener further includes a body disposed within the cavity and in fixed engagement with the inner surface. The body includes a rigid element positioned within only a subregion of the cavity to form a stiffened zone exclusively in an adjacent portion of the shaft.

In one embodiment, the bone fastener includes a shaft having a distal end configured to extend toward and become embedded within bone tissue and a proximal end disposed opposite the distal end. The shaft extends between the proximal end and the distal end to define a longitudinal axis. The shaft further includes an inner surface that defines an axially extending cavity. The bone fastener also includes a core disposed within the cavity and in fixed engagement with the inner surface. The core includes a rigid element selectively positioned within the cavity to form a stiffened zone in an adjacent portion of the shaft. The adjacent portion of the shaft is at least a portion of a distal segment of the shaft extending along the longitudinal axis from a medial portion of the shaft to the distal end. The stiffened zone is configured to have a greater relative stiffness than at least a portion of a proximal segment of the shaft extending along the longitudinal axis from the distal segment to the proximal end.

In one embodiment, the bone fastener includes a shaft extending from a proximal end to a distal tip and defining a longitudinal axis. The shaft includes an inner surface that defines an axially extending cavity. The bone fastener also includes a head disposed at the proximal end of the shaft and configured for engagement with a construct, and a core disposed within the cavity and in fixed engagement with the inner surface. The core includes a rigid pin selectively positioned within only a subregion of the cavity to form a stiffened zone exclusively in an adjacent portion of the shaft.

In one embodiment, a vertebral construct is provided. The vertebral construct includes at least two bone fasteners, similar to those described herein, and at least one vertebral rod having a first end and a second end. The first end is supported adjacent a proximal end of a first bone fastener and the second end is supported adjacent a proximal end of a second bone fastener.

### BRIEF DESCRIPTION OF THE DRAWINGS

The present disclosure will become more readily apparent from the specific description accompanied by the following drawings, in which:

FIG. 1 is a side view, in cross section, of one particular embodiment of a bone fastener in accordance with the principles of the present disclosure;

FIG. 2 is a side view, in cross section, of the bone fastener shown in FIG. 1, attached to bone in accordance with the principals of the present disclosure;

FIG. 3 is a side view of one particular embodiment of a vertebral construct, including the bone fastener shown in FIG. 1, attached to vertebrae in accordance with the principles of the present disclosure;

FIG. 4 is a plan view of the vertebral construct shown in FIG. 3 attached to vertebrae;

FIG. 5 is a side view, in cross section, of one embodiment of the bone fastener shown in FIG. 1;

FIG. 6 is a side view, in cross section, of one embodiment of the bone fastener shown in FIG. 1;

FIG. 7 is a side view, in cross section, of one embodiment of the bone fastener shown in FIG. 1;

FIG. 8 is a side view, in cross section, of one embodiment of the bone fastener shown in FIG. 1;

FIG. 9 is a side view, in cross section, of one embodiment of the bone fastener shown in FIG. 1;

FIG. 10 is a side view, in cross section, of one embodiment of the bone fastener shown in FIG. 1;

FIG. 11 is a side view, in cross section, of one embodiment of the bone fastener shown in FIG. 1;

FIG. 12 is a side view, in cross section, of one embodiment of the bone fastener shown in FIG. 1;

FIG. 13 is a side view, in cross section, of one embodiment of the bone fastener shown in FIG. 1; and

FIG. 14 is a side view, in cross section, of one embodiment of the bone fastener shown in FIG. 1.

Like reference numerals indicate the same or similar parts throughout the figures.

### DETAILED DESCRIPTION OF THE INVENTION

The exemplary embodiments of the bone fastener and methods of use disclosed are discussed in terms of medical devices for the treatment of bone disorders and more particularly, in terms of a bone fastener that includes a flexible component configured to minimize stress to a bone construct and adjacent tissues, including bone, for applications such as, for example, a vertebral rod system. The flexible component of the bone fastener facilitates motion to prevent fastener failure, which may include fastener fracture and/or loosening. It is envisioned that employment of the bone fastener with a vertebral rod system provides stability and maintains structural integrity while reducing stress on spinal elements. The flexible bone fastener may also be used with other constructs such as plates. It is contemplated that a bone construct may include the bone fastener only, in for example, fracture repair such as femur and arthroscopy applications.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures, of such disorders, such as, for example, discectomy, laminectomy, fusion, bone graft and/or implantable prosthetics. It is contemplated that the present disclosure may be employed with other osteal and bone related applications, including those associated with diagnostics and therapeutics. It is further contemplated that the disclosed bone fastener may be employed in a surgical treatment with a patient in a prone or supine position, employing a posterior, lateral or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

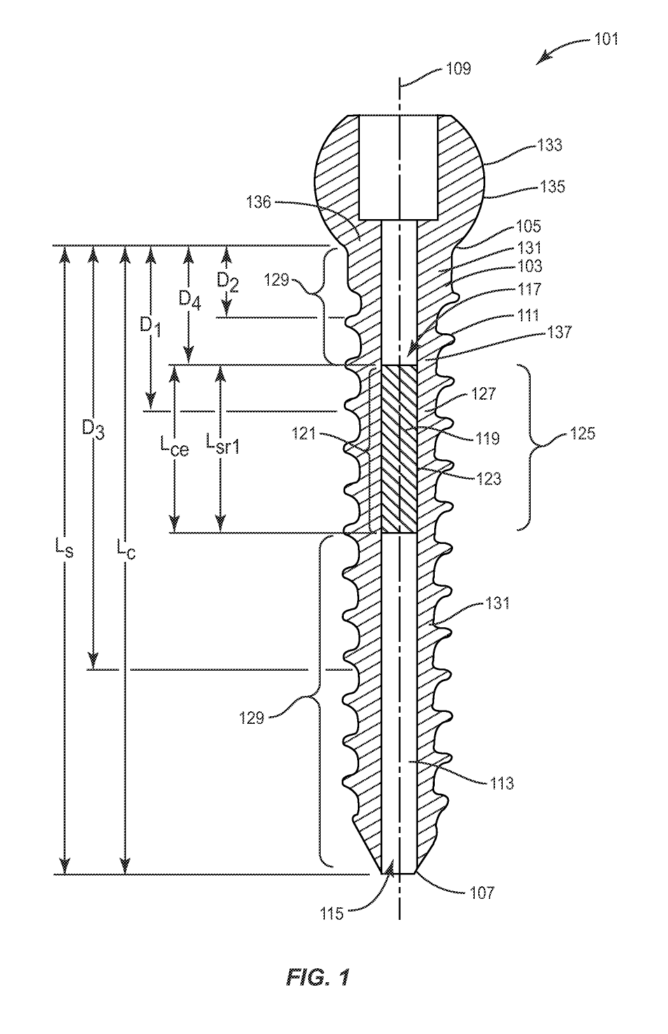

The following discussion includes a description of a bone fastener, related components and exemplary methods of employing the bone fastener in accordance with the principles of the present disclosure. Alternate embodiments are also disclosed. Reference will now be made in detail to the exemplary embodiments of the present disclosure, which are illustrated in the accompanying figures. Turning now to FIG. 1, there is illustrated components of a bone fastener 101 in accordance with the principles of the present disclosure.

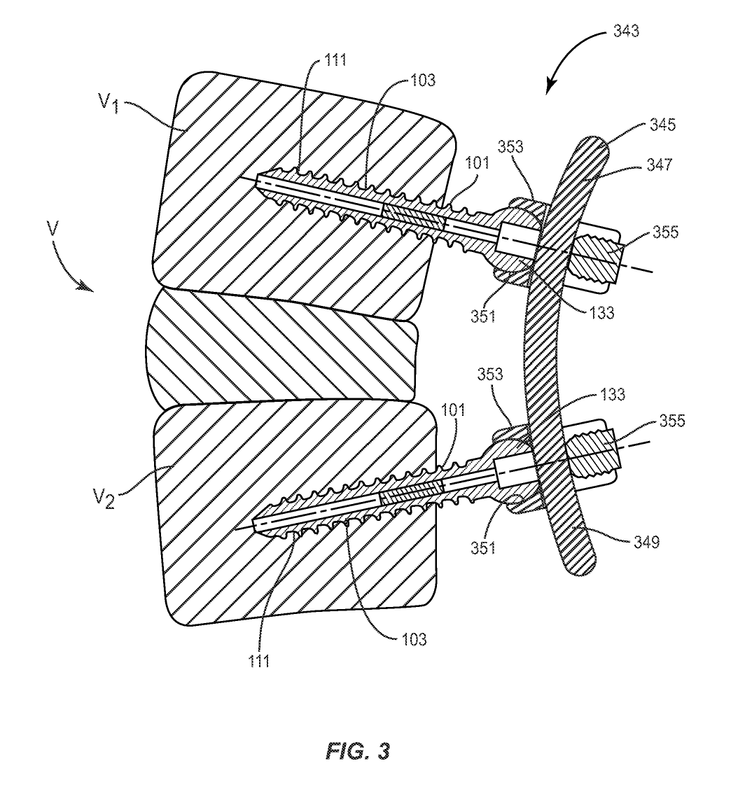

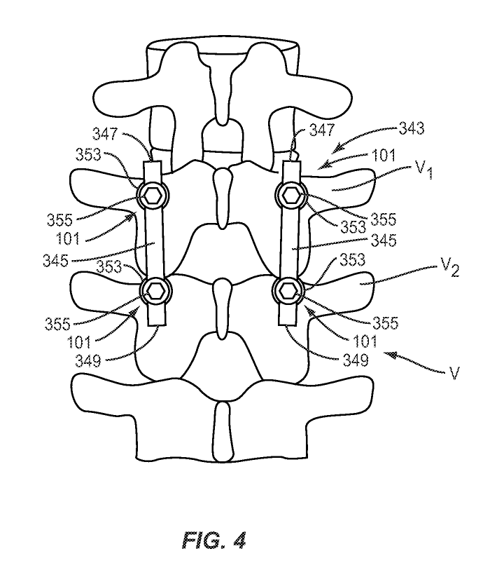

The components of the bone fastener 101 and bone constructs, such as, for example, a vertebral rod system (see, for example, FIGS. 3 and 4) with which the bone fastener 101 may be employed, are fabricated from materials suitable for medical applications, including metals, polymers, ceramics, biocompatible materials and/or their composites, depending on the particular application and/or preference of a medical practitioner. For example, at least one or more, or each, of the components of the bone fastener 101 and/or a vertebral rod (discussed below), of the vertebral rod system (discussed below) may be fabricated from materials such as commercially pure titanium, titanium alloys, Grade 5 titanium, super-elastic titanium alloys, cobalt-chrome alloys, stainless steel alloys, superelastic metallic alloys (e.g. Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof such as calcium phosphate (e.g. SKELITE™ manufactured by Biologix Inc.), rigid polymers including polyphenylene, polyamide, polyimide, polyetherimide, polyethylene, polyurethanes of any durometer, epoxy and silicone. Different components of the bone fastener 101 and/or the vertebral rod system (discussed below) may have alternative material composites to achieve various desired characteristics such as strength, rigidity, elasticity, compliance, biomechanical performance, durability and radiolucency or imaging preference. The components of the bone fastener 101 and/or the vertebral rod system may also be fabricated from a heterogeneous material such as a combination of two or more of the above-described materials.

It is envisioned that the components of the bone fastener 101 and/or the vertebral rod system (see, for example, FIGS. 3 and 4) can be manufactured via various methods. For example, bone fastener 101 can be manufactured and assembled via injection-molding, insert-molding, overmolding, compression molding, transfer molding, co-extrusion, pultrusion, dip-coating, spray-coating, powder-coating, porous-coating, machining, milling from a solid stock material, and their combinations. One skilled in the art, however, will realize that other materials and fabrication methods suitable for assembly and manufacture in accordance with the present disclosure would be appropriate.

The bone fastener 101 may be employed with a vertebral rod system (see, for example, FIGS. 3 and 4) that is configured for attachment to bone tissue, such as, for example, the bone tissue of one or more vertebrae (discussed below) during surgical treatment of a spinal disorder, examples of which are discussed herein. The bone fastener 101 may have increased durability compared to conventional bone screws, at least in that the bone fastener 101 provides an improved fastener/bone interface while maintaining the same mechanical performance in modes of testing such as axial grip, ball slip, etc. The design and fabrication concepts associated with the bone fastener 101 disclosed herein may be applied to multi-axis screws (MAS), fixed axis screws (FAS) and/or posted screws.

Bone fastener 101 includes an outer component or shaft 103 having a first or proximal end 105 and a second or distal end 107. The shaft 103 extends from a proximal segment including the proximal end 105 to a distal segment including the distal end 107, defining a longitudinal axis 109. The shaft 103 has an overall length extent Lₛ along the longitudinal axis 109. The shaft 103 includes a threaded outer surface 111 and an inner surface 113 that defines an axially extending, cylindrically shaped cavity 115. The cavity 115 extends from the proximal end 105 of the shaft 103 to the distal end 107 of the shaft 103. As such, the cavity 115 has an overall length extent L_{c} along the longitudinal axis 109 within the shaft 103 equivalent to the overall length extent Lₛ of the shaft 103. The shaft 103 may be in the form of a shaft featuring the threaded outer surface 111 and fabricated from a compliant material such as PEEK to improve the distribution of stress at the fastener/bone interface.

The bone fastener 101 further includes an inner component or body 117 disposed within the cavity 115 and in fixed engagement with the inner surface 113. The inner body 117 includes a core element 119. The core element 119 has a length extent L_{ce} along the longitudinal axis 109 and is disposed within only a first subregion 121 of the cavity 115. The first subregion 121 of the cavity 115 has a length extent Lₛᵣ₁ along the longitudinal axis 109. The core element 119 includes a cylindrically shaped outer surface 123 in fixed engagement with the inner surface 113 of the cavity 115 continuously across the entire length extent L_{ce} of the core element 119. In another embodiment, the engagement between the outer surface 123 and the inner surface 113 may be varied. For example, the outer surface 123 may be threaded to allow insertion of the core to specific depths. The area of engagement between the outer surface 123 of the core element 119 and the inner surface 113 of the cavity 115 defines the first subregion 121 of the cavity 115. The length extent L_{ce} of the core element 119 and the length extent Lₛᵣ₁ of the first subregion 121 are equivalent.

The material of the core element 119 is continuous across its entire length extent L_{ce}, such that the core element 119 is of monolithic construction. For example, the core element 119 may consist of a rigid cylindrical pin made of solid stainless steel or titanium or another strong, high density material such as a ceramic material. The continuous fixed engagement between the outer surface 123 of the core element 119 and the inner surface 113 of the cavity 115 and the monolithic construction of the core element 119 combine to form a stiffened zone 125 of the bone fastener 101 exclusively in a first portion 127 of the shaft 103 adjacent to the first subregion 121 of the cavity 115. Each of the length extent (not separately indicated) of the stiffened zone 125 of the bone fastener 101 along the longitudinal axis 109 and the length extent (not separately indicated) of the first portion 127 of the shaft 103 along the longitudinal axis 109 is equivalent to the length extent L_{ce} of the core element 119 and the length extent Lₛᵣ₁ of the first subregion 121 of the cavity 115.

The area of the inner surface 113 of the cavity 115 where the inner surface 113 is not engaged with the core element 119 forms or defines a second subregion 129 of the cavity 115 consisting of empty space. A second portion 131 of the shaft 103 adjacent to the second subregion 129 of the cavity 115 is not directly supported or reinforced by the core element 119. The bone fastener 101 has a greater relative stiffness within the stiffened zone 125 than it has along a remaining portion or portions of the shaft 103 outside the stiffened zone 125 (such as, for example, along the second portion 131 of the shaft 103). For example, the bone fastener 101 has a greater relative stiffness at a first distance D₁ from the proximal end 105 of the shaft 103 within the stiffened zone 125 than it has at a distance D₂ from the proximal end 105 within the second portion 131 of the shaft 103 that is shorter than the distance D₁. For another example, the bone fastener 101 has a greater relative stiffness at the first relative distance D₁ from the proximal end 105 within the stiffened zone 125 than it has at a distance D₃ from the proximal end 105 within the second portion 131 of the shaft 103 that is longer than the distance D₁.

The length L_{ce} of the core element 119 is a fraction of the overall length extent Lₛ of the shaft 103. The length L_{ce} of the core element 119 is based upon the length of a pedicle of the vertebra. The length of the core element 119 may extend along the length of the shaft 103 for the amount the shaft 103 extends through the pedicle.

A distance D₄ between the proximal end of the shaft 103 and the stiffened zone is a fraction of the overall length extent Lₛ of the shaft 103. It is contemplated that distance D₄ between the proximal end of the shaft and the stiffened zone may be, such as, for example 2 mm to 25 mm.

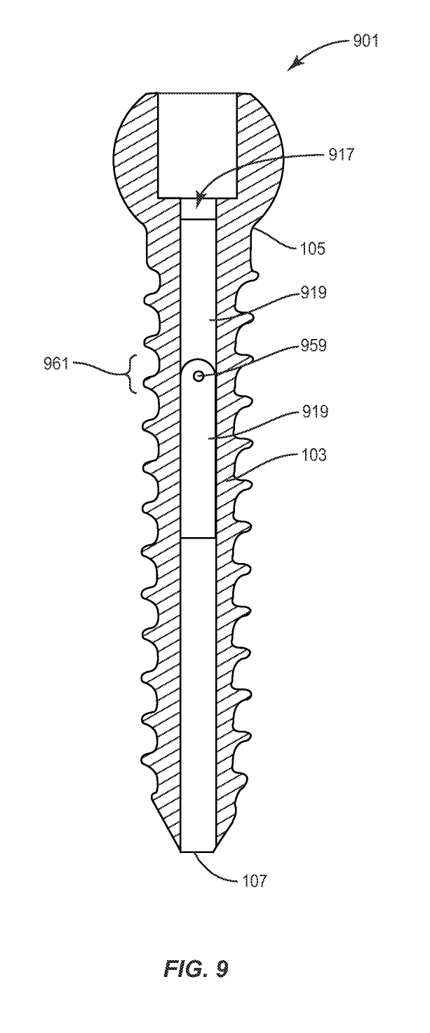

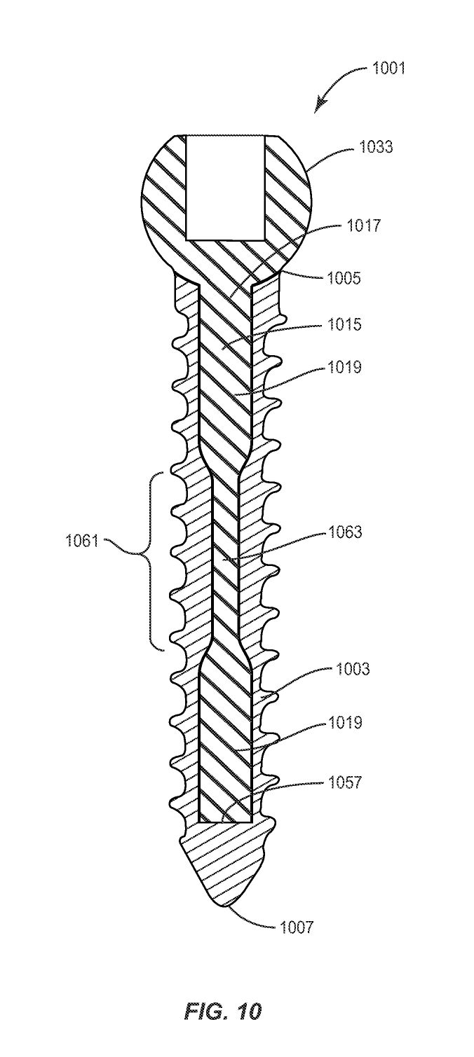

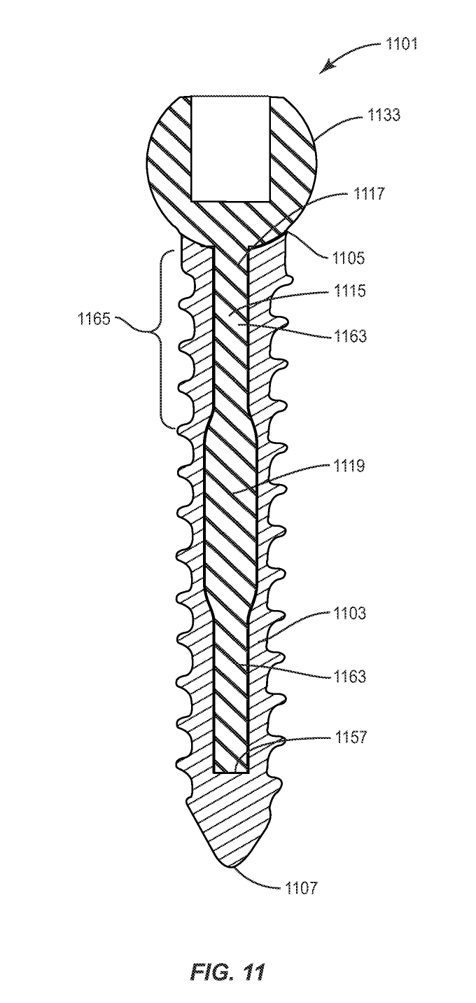

Various embodiments of the bone fastener 101 described herein include an axially elongated outer component or shaft 103 including an axially extending cavity 115 and an axially elongated core element 119 positioned within only a subregion 121 of the cavity 115 to form a stiffened zone 125 exclusively in an axial portion 127 of the shaft 103 adjacent to the subregion 121 of the cavity 115 occupied by the axially elongated core element 119. For example, in one embodiment the core element 119 has a different material property such as strength modulus, and flexibility relative to the shaft 103. In one embodiment, particular parameters of the fabrication material of the shaft 103 are selected to increase the flexibility or decrease the stiffness of the bone fastener 101 outside the stiffened zone 125 including the material modulus that may correlate to hardness and modification of porosity, which may include modification of void volume. In one embodiment, the core element 119 is positioned at some depth from a proximal end 105 of the shaft 103 such that a zone of relative flexibility is formed in the bone fastener between the proximal end 105 of the shaft 103 and the stiffened zone 125. In one embodiment, the core element 119 is positioned entirely between the proximal end 105 and the distal end 107 of the shaft 103, and at the proximal end 105 of the shaft 103, the bone fastener 111 includes a head 133 configured and dimensioned for engagement with a spinal construct. In one embodiment, the core element 119 can be selectively positioned at one of multiple different positions within the cavity 115 along the longitudinal axis 109, including one or more axial positions at or near the proximal end 105 of the shaft 103, and one or more axial positions at or near the distal end 107 of the shaft 103. In one embodiment, the inner body 117 includes at least one further part (not shown, see, for example, FIGS. 10 and 11) configured differently than the core element 119 and positioned within a different subregion of the cavity (not specifically indicated) such that the bone fastener 101 has greater flexibility in such different subregion of the cavity than within the stiffened zone 125. In one embodiment, the core element 119 is disposed between the proximal end 105 of the shaft 103 and the distal end 107 of the shaft 103 and includes a first axially elongated part (not shown, see, for example, FIGS. 9 and 10) and a second axially elongated part (not shown, see, for example, FIGS. 9 and 10) distal the first axially elongated part such that the bone fastener 101 has a greater flexibility in a portion (not specifically indicated) of the shaft 103 between the first and second axially elongated parts of the core element 119 than within the stiffened zone 125. In one embodiment, the core element 119 is disposed between the proximal end 105 of the shaft 103 and the distal end 107 of the shaft 103 and includes a first axially elongated part adjacent the first axial end (not shown, see, for example, FIGS. 9 and 10) and a second axially elongated part adjacent the second axial end (not shown, see, for example, FIGS. 9 and 10), and the inner body 117 includes a flexible hinge (not shown, see, for example, FIGS. 9 and 10) disposed between the two parts of the core element such that the bone fastener 101 has a greater flexibility in a portion of the shaft 103 between the first and second axially elongated parts of the core element 119 than within the stiffened zone 125.

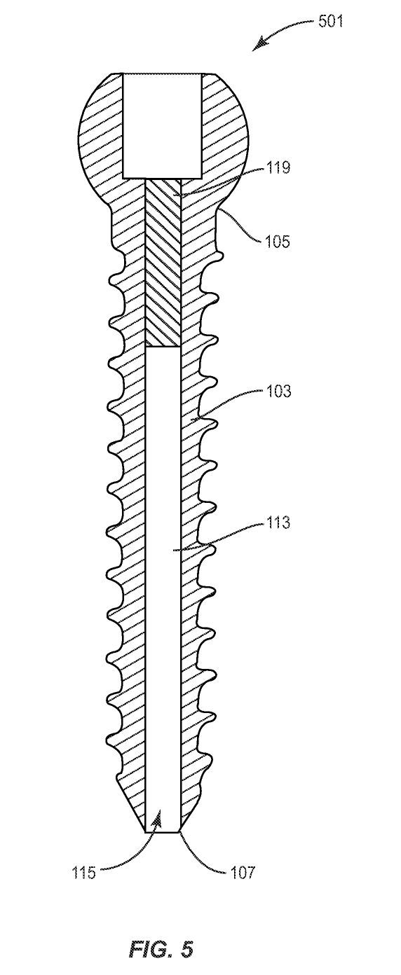

FIG. 1 shows the second portion 131 of the shaft 103, representing the portion or portions of the shaft 103 that is not reinforced by the core element 119, as being divided between two parts or portions separated from each other by the first portion 127 of the shaft 103 that is reinforced by the core element 119. It is contemplated that the second portion 131 of the shaft 103 may consist of only one portion of the shaft 103 (such as, for example, is shown in FIGS. 5, 6, 7, and 8). Other arrangements are possible, including arrangements in which the second portion 131 is divided into three or more separate portions.

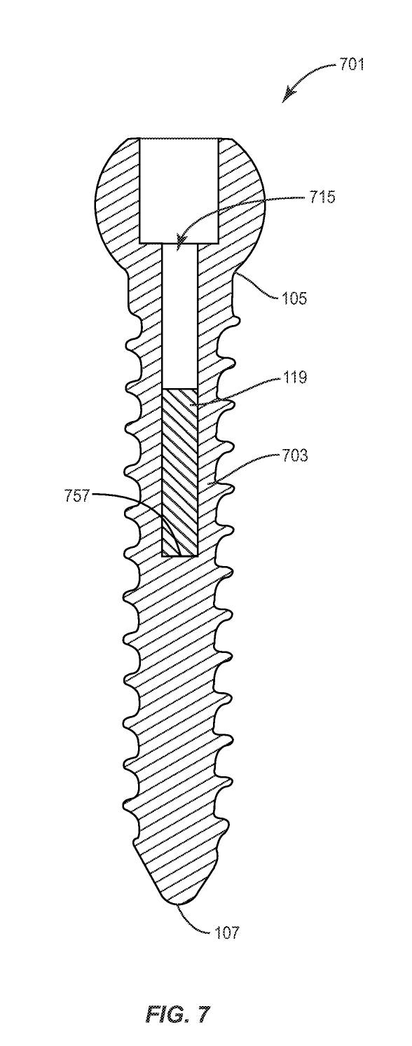

Cavity 115 passes entirely through the shaft 103. It is contemplated that the cavity 115 may stop short of passing entirely through the shaft 103. For example, the cavity 115 may be or form a blind hole, and/or be closed at or adjacent to one or both of the proximal end 105 and the distal end 107 of the shaft 103 (such as, for example, is shown in FIGS. 7, 8, 10, 11, 12, 13, 14).

FIG. 1 shows the core element 119 of the inner body 117 having only one part. It is contemplated that the core element 119 of the inner body 117 may include two parts, each part separated from the other (such as, for example, is shown in FIGS. 9 and 10). Other arrangements are possible, including embodiments in which the core element 119 of the inner body 117 includes three or more parts, each part separated from the other.

FIG. 1 shows the inner body 117 including no structure other than the core element 119. It is contemplated that the inner body 117 may include structure other than the core element 119. For example, it is contemplated that the inner body 117 may further include one or more hinges (such as, for example, is shown in FIGS. 9 and 10). It is further contemplated that the inner body 117 may include one or more arms or extensions adjacent to the core element 119 having relatively reduced or smaller cross-sectional dimensions so as to offer relatively less stiffening or reinforcing power vis-a-vis the shaft 103 (such as, for example, is shown in FIGS. 10 and 11). Other configurations are possible.

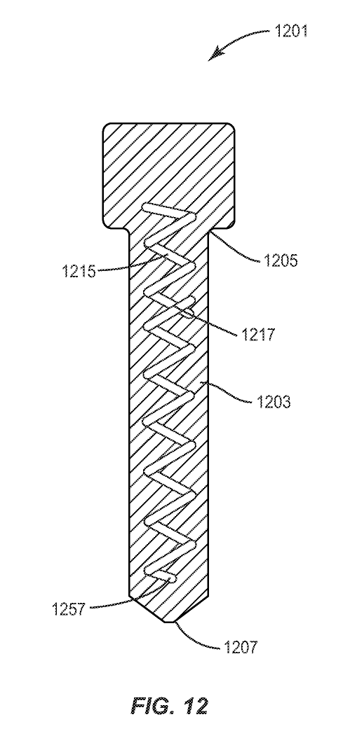

FIG. 1 shows the inner body 117 or the core element 119 having an axially straight geometry along the longitudinal axis 109. It is contemplated that the inner body 117 and/or the core element 119 may have geometries other than straight axial geometries. For example, the inner body 117 and/or the core element 119 may have an axially extending helical geometry (such as, for example, is shown in FIG. 12).

FIG. 1 shows the inner body 117 or the core element 119 having a cross-sectional geometry that is consistent along the direction of the longitudinal axis 109. It is contemplated that the inner body 117 and/or the core element 119 may have a cross-sectional geometry that varies, either relatively smoothly (e.g., with large radius curves), relatively abruptly (e.g., with short radius curves, and/or with angles or in steps), or a combination thereof, along the direction of the longitudinal axis 109 (such as, for example, is shown in FIGS. 10, 11, 13, and 14).

FIG. 1 shows the cavity 115 including empty space. It is contemplated that the cavity may be substantially and/or completely occupied by the inner body 117 and/or the core element 119 (such as, for example, is shown in FIGS. 9, 10, and 11).

FIG. 1 shows the outer surface 111 as being threaded. It is contemplated that the outer surface may be partially or completely unthreaded. For example, in at least some embodiments (not specifically shown), the outer surface 111 of the bone fastener 101 is smooth and substantially cylindrical in shape, and the bone fastener 101 is a reinforced bone fixation pin or rod.

The material of the core element 119 is continuous across its entire length extent L_{ce}, such that the core element 119 is of monolithic construction. It is contemplated that the core element 119 may be non-monolithic. For example, the core element 119 may be fabricated from a material having intermittent gaps or voids, and/or may be a multi-part assembly.

As indicated above, the bone fastener 101 includes a head 133 configured and dimensioned for engagement with a spinal construct. The head 133 includes a spherical outer surface 135 that allows the head 133 to interact with a socket portion of a ball-and-socket joint (not shown, see FIG. 3), and/or to support a vertebral rod (not shown, see FIG. 3), at least indirectly. It is contemplated that the outer surface 135 of the head 133 may be non-spherical, and/or that the head 133 may be configured and dimensioned to directly support a vertebral rod (not shown). Other configurations are possible. For example, in at least some embodiments of the bone fastener 101 in accordance with the principles of the present disclosure, no separate head is provided adjacent to the proximal end 105 of the shaft 103, such that the overall length extent of the bone fastener 101 along the longitudinal axis 109 is equivalent to the overall length extent Lₛ of the shaft 103.

FIG. 1 shows the head 133 and the shaft 103 as two different portions of a single component of unitary construction. In such circumstances, the single component of which the head 133 and the shaft 103 comprise two different portions may be described as a screw 136. It is envisioned that the head 133 and the shaft 103 may comprise two parts of a multi-part component or assembly in which the head 133 and the shaft 103 are directly coupled to each other (not separately shown). It is further envisioned that the head 133 and the shaft 103 may be indirectly coupled to each other. For example, it is contemplated that instead of being of unitary construction with the shaft 103, the head 133 may be of unitary construction with the inner body 117 and/or the core element 119 (as shown, for example, in FIGS. 10 and 11).

It is contemplated that the core element 119 or only portions thereof can be variously dimensioned, for example, with regard to length, width, diameter and thickness. It is further contemplated that the cross-sectional geometry or only portions thereof may have various configurations, for example, round, oval, rectangular, irregular, consistent, variable, uniform and non-uniform.

It is envisioned that the core element 119 may include alternate locking or fastening elements to fix the core element 119 with the shaft 103 such as integral connection, threaded engagement, clips, friction fit, interference fit, pins and/or adhesive.

The cavity 115 is configured for disposal of the core element 119. The inner surface 113 has a continuous, non-interrupted configuration and is disposed in close fitting engagement with the outer surface 123. It is contemplated that the inner surface 113 may be non-continuous and interrupted, such as, for example, slotted, perforated, dimpled and/or undulating.

The inner surface 113 and the outer surface 111 define a wall 137 having a thickness and that is circumferentially disposed about the entire outer surface 123 of the core element 119. It is contemplated that the shaft 103 can be variously dimensioned, for example, with regard to the length or thickness of the wall 137, and cross sectional geometry such as those discussed above. For example, the cross-sectional geometries of the outer surface 111 and/or the inner surface 113 can be round, oval, rectangular, irregular, consistent, variable, uniform and non-uniform, and surfaces 111, 113 may have the same or different cross section geometry.

As indicated above, the outer surface 111 of the shaft 103 is threaded for fixation with bone. It is contemplated that the shaft 103 may include alternate bone fixation elements, such as, for example, a nail configuration, barbs, and/or expanding elements. The shaft 103 may have a different cross-sectional area, geometry, material or material property such as strength, modulus or flexibility relative to the core element 119.

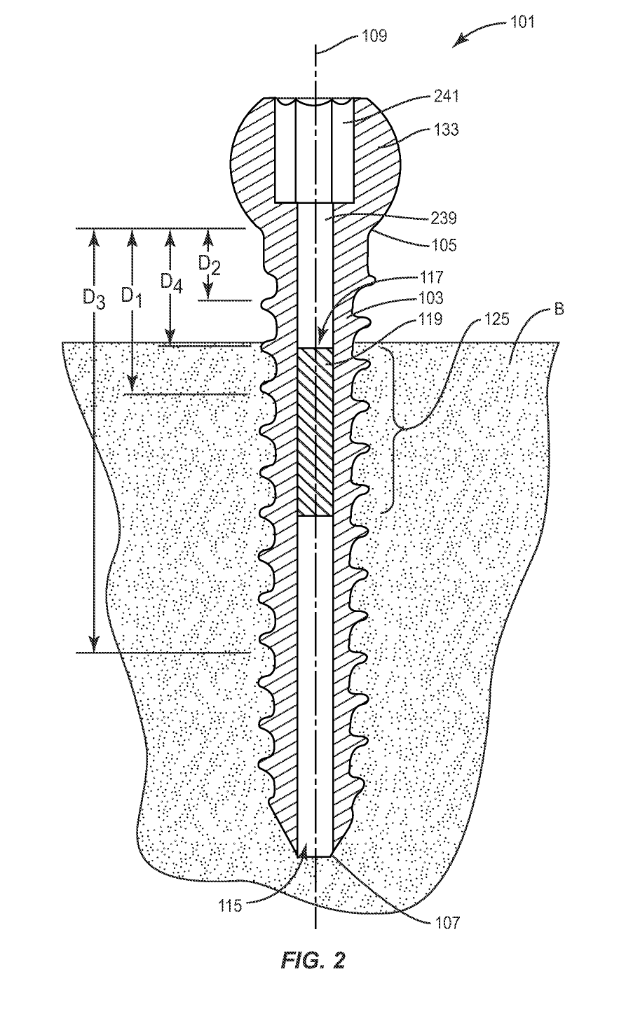

Turning now to FIG. 2, in assembly, operation and use, the bone fastener 101 is employed with a surgical procedure for treatment of a spinal disorder affecting a section of a spine of a patient, as discussed herein. The bone fastener 101 may also be employed with other surgical procedures. Prior to implantation of the bone fastener 101 in bone tissue B, a practitioner inserts the inner body 117, which in this example consists of the core element 119, into the cavity 115, either through a complementary cavity 239 formed in the head 133 and into the shaft 103 through the proximal end 105 thereof, or into the shaft 103 through the distal end 107 thereof. When the core element 119 is in the position within the shaft 103 and along the longitudinal axis 109 desired by the practitioner, the practitioner stops moving the core element 119 relative to the shaft 103 and fixes the former in place relative to the latter within the cavity 115 using any conventional attachment means, including but not necessarily limited to friction fit, interference fit, and/or fast-setting adhesive.

When ready, the practitioner directs the distal end 107 of the shaft 103 toward the bone tissue B and implants the bone fastener 101 into the bone tissue B to a selected or predetermined depth. For this purpose, the practitioner utilizes a hex socket 241 formed in a proximal side of the head 133 to rotate and drive the bone fastener 101 into the bone tissue B. As an example of one depth the practitioner may select, the entire stiffened zone 125 of the bone fastener 101 is embedded within the bone tissue B. As shown, in this configuration, the relatively stiff portion of the bone fastener 101 corresponding to depth D₁ below the proximal end 105 within the stiffened zone 125 is contained within the bone tissue B, while the relatively flexible portion of the bone fastener 101 corresponding to depth D₂ below the proximal end 105 is positioned above the surface of the bone tissue B.

The composite design of the bone fastener 101 below the surface of the bone tissue B is advantageous in that the threaded shaft 103 is made of a compliant material such as PEEK to improve the distribution of the stress at the fastener/bone interface. In addition, the shape, material, and relative position of the core element 119 improves the durability of the bone fastener 101 against the distributed forces on the fastener/bone interface from the fixation of the vertebrae (not shown), and reduces the chance of disassembly of the bone fastener 101 during screwing or unscrewing of the bone fastener 101 in bone and during axial loading of the shaft 103 (e.g., during pull-out testing).

The relative flexibility of the bone fastener 101 above the surface of the bone tissue B near the proximal end 105 of the shaft 103 and adjacent portions of the head 133 performs another beneficial stress distribution function, e.g., with respect to forces imposed upon the bone fastener 101 by adjacent hardware of the spinal construct (not shown, see FIGS. 3 and 4), while maintaining the same mechanical performance in the modes of testing such as axial grip and ball slip.

In a first orientation of the bone fastener 101, both the head 133 and the core element 119 are longitudinally aligned with the shaft 103 along longitudinal axis 109. It is contemplated that in the first orientation, no flexion forces are applied to the bone fastener 101. As the components of the bone fastener 101 move to a second orientation from the first orientation, flexion forces are applied to the bone fastener 101 such that the head 133 and the adjacent portion of the shaft 103 are at least partially pivotable together to a plurality of axial orientations relative to the longitudinal axis 109 of the shaft 103. It is envisioned that such relative pivotable movement of the head 133 and the adjacent portion of the shaft 103 includes bending through angle α relative to the longitudinal axis 109.

Turning now to FIGS. 3 and 4, a vertebral rod system 343 including multiple instances of the bone fastener 101 and multiple vertebral rods 345 is employed with a surgical procedure for treatment of a condition or injury of an affected section of the spine including vertebrae V. It is contemplated that the vertebral rod system 343 including the bone fastener 101 is attached to vertebrae V for fusion applications of the affected section of the spine to facilitate healing and therapeutic treatment.

In use, to treat the affected section of the spine, a medical practitioner obtains access to a surgical site including vertebra V in any appropriate manner, such as through incision and retraction of tissues. It is envisioned that the vertebral rod system 343 including the bone fastener 101 may be used in any existing surgical method or technique including open surgery, mini-open surgery, minimally invasive surgery and percutaneous surgical implantation, whereby vertebrae V is accessed through a micro-incision, or sleeve that provides a protected passageway to the area. Once access to the surgical site is obtained, the particular surgical procedure is performed for treating the spinal disorder. The vertebral rod system 343 including the bone fastener 101 is then employed to augment the surgical treatment. The vertebral rod system 343 including the bone fastener 101 can be delivered or implanted as a pre-assembled device or can be assembled in situ. The vertebral rod system 343 may be completely or partially revised, removed or replaced, for example, replacing one or both of the vertebral rods 345 and/or one or all of the components of the bone fastener 101.

The vertebral rod 345 has a rigid, arcuate configuration. A first bone fastener 101 is configured to attach an upper section 347 of the vertebral rod 345 to vertebra V₁. A second bone fastener 101 is configured to attach a lower section 349 of the vertebral rod 12 to adjacent vertebra V₂. Pilot holes are made in the vertebrae V₁, V₂ for receiving the first and second bone fasteners 101. Each shaft 103 of the first and second bone fasteners 101 includes the threaded bone engaging outer surface 111 that is inserted or otherwise connected to vertebrae V₁, V₂, according to the particular requirements of the surgical treatment. Each head 133 of the first and second bone fasteners 101 is inserted into a corresponding socket 351 of a coupling element 353 configured to support the vertebral rod 345, and a set screw 355 is torqued onto the sections 347, 349 to attach the vertebral rod 345 in place with vertebrae V. It is envisioned that the vertebral rod 345 may have a semi-rigid or flexible configuration.

As shown in FIG. 4, the vertebral rod system 343 includes two axially aligned and spaced vertebral rods 345, with sections 347, 349 extending over or past the heads 133 of the bone fasteners 101 (see FIG. 3). The set screws 355 of each coupling element 353 are torqued on the end portions of the vertebral rods 345 to securely attach the vertebral rods 345 with vertebrae V₁, V₂.

The bone fastener 101 may be employed as a bone screw, pedicle screw or MAS used in spinal surgery. It is contemplated that bone fastener 10 may be coated with an osteoconductive material such as hydroxyapatite and/or osteoinductive agent such as a bone morphogenic protein for enhanced bony fixation. The bone fastener 101 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used, such as being disposed at the end portions of the vertebral rod 345.

It is envisioned that the vertebral rod system described above including the bone fastener 101 may be employed with a vertebral rod 345 having an arcuate configuration and an increased length providing the ability to extend over two or more intervertebral elements. It is contemplated that the configuration of the vertebral rod system 343 may provide load sharing and/or stabilization over a plurality of intervertebral levels, including treated and untreated vertebral and intervertebral levels.

In one embodiment, as shown in FIG. 5, a bone fastener 501, similar to bone fastener 101 described above, includes the core element 119 disposed within the cavity 115 in fixed engagement with the inner surface 113 of the shaft 103 at the proximal end 105 thereof.

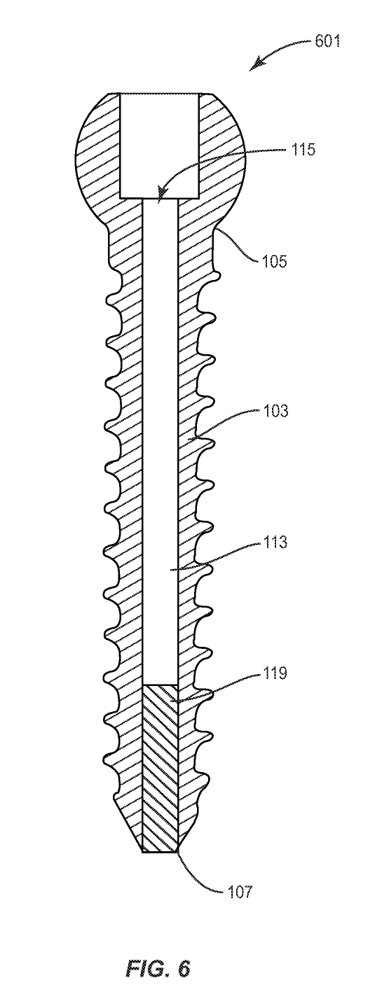

In one embodiment, as shown in FIG. 6, a bone fastener 601, similar to bone fastener 101 described above, includes the core element 119 disposed within the cavity 115 in fixed engagement with the inner surface 113 of the shaft 103 at the distal end 107 thereof.

In one embodiment, as shown in FIG. 7, a bone fastener 701, similar to bone fastener 101 described above, includes a shaft 703 including a cavity 715 that does not extend the entire distance from the proximal end 105 to the distal end 107 of the shaft 703, but rather terminates short of the distal end 107 at a wall 757 within the shaft 703 and, as such, constitutes a blind hole.

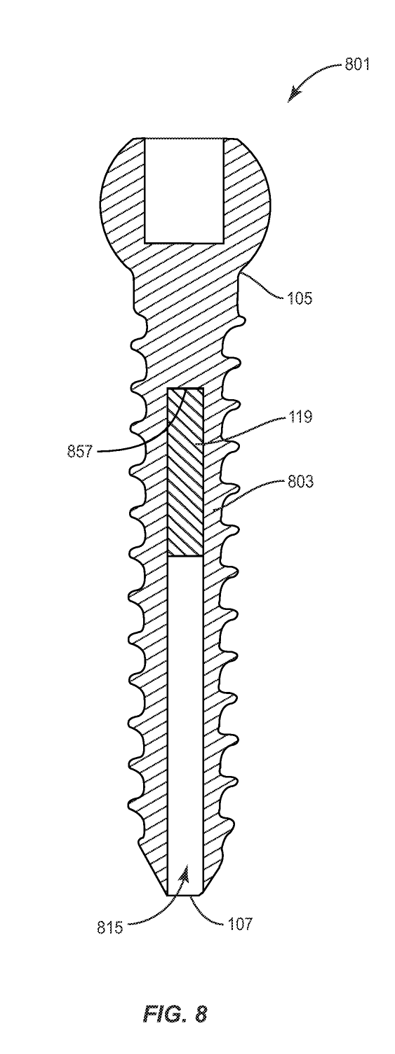

In one embodiment, as shown in FIG. 8, a bone fastener 801, similar to bone fastener 101 described above, includes a shaft 803 including a cavity 815 that does not extend the entire distance from the distal end 107 to the proximal end 105 of the shaft 803, but rather terminates short of the proximal end 105 at a wall 857 within the shaft 803 and, as such, constitutes a blind hole.

In one embodiment, as shown in FIG. 9, a bone fastener 901, similar to bone fastener 101 described above, includes an inner body 917 that has two core elements 919. Inner body 917 includes a flexible hinge 959 disposed in the cavity 115 between the core elements 919 and adjacent a medial portion 961 of the shaft 103 between the proximal end 105 and the distal end 107 thereof. Hinge 959 is a U-joint. It is contemplated that the hinge 959 may be or include another type of joint, such as, for example, a ball joint, or a living hinge (such as, for example, is shown in FIGS. 10 and 11). Other configurations for the hinge 959 are possible.

In one embodiment, as shown in FIG. 10, a bone fastener 1001, similar to bone fastener 101 described above, includes a shaft 1003 having a cavity 1015 that does not extend the entire distance from the proximal end 1005 to the distal end 1007 of the shaft 1003, but rather terminates short of the distal end 1007 at a wall 1057 within the shaft 1003 and, as such, constitutes a blind hole. The head 1033 of the bone fastener 1001 is of unitary construction with the inner body 1017. The cavity 1015 has a variable diameter along its axial length (e.g., to conform to a corresponding dimension of the inner body 1017). The inner body 1017 of the bone fastener 1001 has two core elements 1019. The inner body 1017 further includes an axially elongated portion 1063 having a reduced diameter (e.g., as compared to the core elements 1019) disposed in the cavity 1015 between the core elements 1019, and that functions in the manner of a living hinge between the core elements 110 such that the inner body 1017 is articulated. The reduced diameter portion 1063 is disposed in the cavity 1015 adjacent a medial portion 1061 of the shaft 1003 between the proximal end 1005 and the distal end 1007 thereof. The head 1033 of the bone fastener 1001 is of unitary construction with the inner body 1017 and/or with one of the core elements 1019.

In one embodiment, as shown in FIG. 11, a bone fastener 1101, similar to bone fastener 101 described above, includes a shaft 1103 having a cavity 1115 that does not extend the entire distance from the proximal end 1105 to the distal end 1107 of the shaft 1103, but rather terminates short of the distal end 1107 at a wall 1157 within the shaft 1103 and, as such, constitutes a blind hole. The head 1133 of the bone fastener 1101 is of unitary construction with the inner body 1117. The cavity 1115 has a variable diameter along its axial length (e.g., to conform to a corresponding dimension of the inner body 1117). The inner body 1117 of the bone fastener 1001 has two axially elongated, reduced diameter portions 1163 disposed in the cavity 1115 adjacent the core element 1119. One of the two reduced diameter portions 1163 is disposed in a proximal portion 1165 of the shaft 1103 adjacent the proximal end 1105 thereof. The reduced diameter portion 1163 disposed in the proximal portion 1165 of the shaft 1103 functions in the manner of a living hinge between the head 1133 and the core element 1119.

In one embodiment, as shown in FIG. 12, a bone fastener 1201, similar to bone fastener 101 described above, includes a shaft 1203 having a cavity 1215 that does not extend the entire distance from the proximal end 1205 to the distal end 1207 of the shaft 1203, but rather terminates short of the distal end 1207 at a wall 1257 within the shaft 1203 and, as such, constitutes a blind hole. The inner body 1217 has an axially extending helical geometry. The inner body 1217 is a spring core. The spring core formed by the helical geometry of the inner body 1217 has a uniform geometry along its axial length. It is contemplated that the spring core formed by the helical geometry of the inner body 1217 may have a non-uniform, and/or variable, geometry along its axial length, including, for example, both large and small diameter coils, and/or axially elongated sections characterized by different pitch parameters (e.g., relatively large or small pitch sections). Other configurations are possible. The bone fastener 1201 may be configured for use in surgical applications in which a greater degree of lateral or transverse flexure or deformation is desired in the shaft 1203.

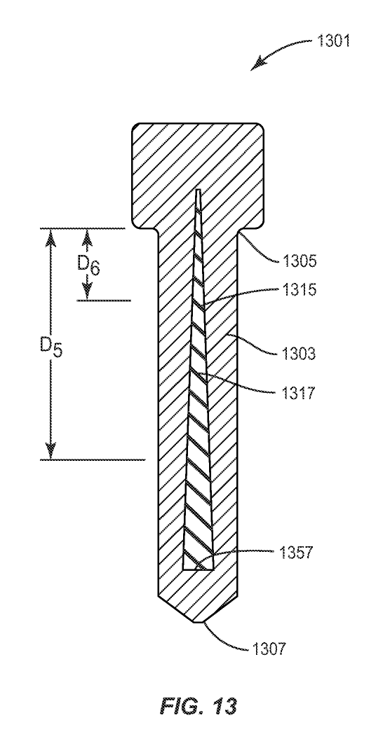

In one embodiment, as shown in FIG. 13, a bone fastener 1301 includes a shaft 1303 having a cavity 1315 that does not extend the entire distance from the proximal end 1305 to the distal end 1307 of the shaft 1303, but rather terminates short of the distal end 1307 at a wall 1357 within the shaft 1303 and, as such, constitutes a blind hole. The inner body 1317 is metallic and has a cross-sectional geometry that varies along its axial length such that the inner body 1317 defines a conical shape. More particularly, the diameter of the inner body 1317 increases as the depth from the proximal end 1305 increases. The lateral or transverse stiffness of the bone fastener 1301 at a large depth D₅ from the proximal end 1305 is relatively greater than the stiffness of the bone fastener 1301 at a relatively small depth D₆ from the proximal end 1305. Because it is equipped with the conically-shaped inner body 1317, the bone fastener 1301 features a longitudinally tapered stiffness.

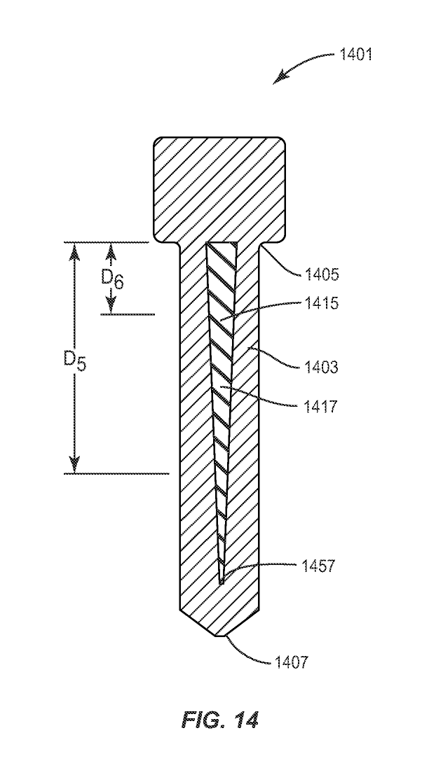

In FIG. 14, a bone fastener 1401 includes a shaft 1403 having a cavity 1415 that does not extend the entire distance from the proximal end 1405 to the distal end 1407 of the shaft 1403, but rather terminates short of the distal end 1407 at a wall 1457 within the shaft 1403 and, as such, constitutes a blind hole. The inner body 1417 is metallic and has a cross-sectional geometry that varies along its axial length such that the inner body 1417 defines a conical shape. More particularly, the diameter of the inner body 1417 decreases, rather than increases, as the depth from the proximal end 1405 increases. The lateral or transverse stiffness of the bone fastener 1401 at the small depth D₆ from the proximal end 1405 is relatively greater than the stiffness of the bone fastener 1401 at the relatively large depth D₅ from the proximal end 1405. Because it is equipped with the conically-shaped inner body 1417, the bone fastener 1401 features a longitudinally tapered stiffness.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A bone fastener comprising:
a shaft extending from a first end to a second end and defining a longitudinal axis, the shaft including an inner surface that defines an axially extending cavity; and
a body disposed within the cavity and in fixed engagement with the inner surface, the body including a rigid element positioned within only a subregion of the cavity to form a stiffened zone exclusively in an adjacent portion of the shaft.

2. A bone fastener according to claim 1, wherein the first end of the shaft is configured for engagement with a construct.

3. A bone fastener according to claim 1, wherein the stiffened zone has a greater relative stiffness than a remaining portion of the shaft disposed along and corresponding to the cavity.

4. A bone fastener according to claim 1, wherein the body consists of the rigid element, and a remaining portion of the cavity consists of empty space.

5. A bone fastener according to claim 1, wherein a remaining portion of the shaft disposed along and corresponding to the cavity has a greater relative flexibility than the stiffened zone.

6. A bone fastener according to claim 1, wherein the rigid element is selectively positioned between the first end and the second end.

7. A bone fastener according to claim 1, wherein the stiffened zone is disposed adjacent the second end.

8. A bone fastener according to claim 1, wherein the body includes a first arm hingedly connected to a second arm such that the rigid element includes a first rigid part disposed adjacent the first end and a second rigid part disposed adjacent the second end.

9. A bone fastener according to claim 8, wherein the body includes a flexible hinge disposed between the first arm and the second arm adjacent a medial portion of the shaft.

10. A bone fastener according to claim 9, wherein the hinge is a ball joint.

11. A bone fastener according to claim 9, wherein the hinge is a U-joint.

12. A bone fastener according to claim 9, wherein the hinge is a reduced diameter portion of the body.

13. A bone fastener according to claim 1, wherein the cavity includes a closed end disposed at a medial portion of the shaft between the first end and the second end.

14. A bone fastener according to claim 1, wherein the body consists of the rigid element and the body has an axially extending helical configuration.

15. A bone fastener according to claim 14, wherein the body has an increasing stiffness between the first and second ends.

16. A bone fastener according to claim 1, wherein the body includes an enlarged diameter portion disposed adjacent a medial portion of the shaft such that the rigid element consists of the enlarged diameter portion.

17. A bone fastener comprising:
a shaft having a distal end configured to be embedded within bone tissue and a proximal end disposed opposite the distal end, the shaft extending between the proximal end and the distal end to define a longitudinal axis, the shaft further including an inner surface that defines an axially extending cavity; and
a core disposed within the cavity and in fixed engagement with the inner surface, the core including a rigid element selectively positioned within the cavity to form a stiffened zone in an adjacent portion of the shaft, the adjacent portion being at least a portion of a distal segment of the shaft extending along the longitudinal axis from a medial portion of the shaft to the distal end,
wherein the stiffened zone is configured to have a greater relative stiffness than at least a portion of a proximal segment of the shaft extending along the longitudinal axis from the distal segment to the proximal end.

18. A bone fastener comprising:
a shaft extending from a proximal end to a distal tip and defining a longitudinal axis, the shaft including an inner surface that defines an axially extending cavity;
a head disposed at the proximal end of the shaft and configured for engagement with a construct; and
a core disposed within the cavity and in fixed engagement with the inner surface, the core including a rigid pin selectively positioned within only a subregion of the cavity to form a stiffened zone exclusively in an adjacent portion of the shaft.

19. A bone fastener according to claim 18, wherein the pin is threaded.

20. A bone fastener according to claim 18, wherein the pin is positioned adjacent the head.
